(19) United States
(12) Patent Application Publication
(10) Pub. No.: US 2010/0204779 A1
(43) Pub. Date: Aug. 12, 2010

(54) STENT AND METHOD FOR PRODUCING A STENT

(75) Inventors: Kirsi Schuessler, Pfinztal (DE); Werner Mailaender, Engelsbrand Grunbach (DE)

Correspondence Address: PANITCH SCHWARZE BELISARIO & NADEL LLP, ONE COMMERCE SQUARE, 2005 MARKET STREET, SUITE 2200, PHILADELPHIA, PA, 19103, US

(73) Assignee: ACANDIS GMBH & CO. KG, Pfinztal (DE)

(21) Appl. No.: 12/597,580

(22) PCT Filed: Apr. 28, 2008
(86) PCT No.: PCT/EP08/03436

§ 371 Date: May 3, 2010

(30) Foreign Application Priority Data
Apr. 26, 2007 (DE) 102007019772.3

(51) Int. Cl. A61F 2/82; B23P 11/00

(52) U.S. Cl. 623/1.16; 623/1.2; 29/428

(57) ABSTRACT

A stent with a tubular grid structure (10) includes struts (11) and cell openings (12) formed by the struts (11), wherein in the expanded state of the stent the grid structure has a certain outer diameter D_{A} in mm and the struts each have a certain length L in mm, the outer diameter D_{A} of the stent and the length L of the struts being in a certain ratio.

### CROSS-REFERENCE TO RELATED APPLICATIONS

This application is a section 371 of International Application No. PCT/EP2008/003436, filed Apr. 28, 2008, which was published in the German language on Nov. 6, 2008 under International Publication No. WO 2008/131956 A1 and the disclosure of which is incorporated herein by reference.

### BACKGROUND OF THE INVENTION

The invention relates to a stent having a tubular grid structure which comprises struts and cell openings formed by the struts, wherein in the expanded state of the stent the grid structure has an outer diameter D_{A} in mm and the struts each have a length L in mm. The invention also relates to a process for the production of a stent. Such a stent is known, for example, from U.S. Pat. No. 6,428,569.

Stents formed from a tubular grid structure are employed for expanding and supporting blood vessels. The grid structure comprises struts, which form cell openings, and makes it possible for the stent to be implanted in the folded state by means of a catheter and for the stent to be expanded after the stent has been introduced into the section of vessel to be treated.

The abovementioned U.S. Pat. No. 6,428,569 discloses a stent system which comprises two stents arranged concentrically in one another (stent in stent construction). The two stents each have an essentially continuous sheath surface which is perforated. In the known stent system, the diameter of the effective perforation openings is adjusted by causing the perforation openings of the stent arranged on the inside to partly coincide with the perforation openings of the outer stent. The diameter of the resulting effective perforation opening is increased or reduced according to the degree of overlapping. According to U.S. Pat. No. 6,428,569, perforation openings in the range of from 51 μm to 510 μm can therefore be realized.

In practice, however, it is difficult to impossible to position the two concentrically arranged stents such that a desired degree of overlapping and therefore opening diameter in the abovementioned range are established exactly. Moreover, the wall thickness is correspondingly increased by the stent in stent construction, and the internal lumen of the vessel is reduced. Furthermore, the stent is rigid and is difficult or even impossible to fold up.

U.S. Pat. No. 6,428,569 furthermore discloses a stent with a grid strut structure which is said to have cell openings in the range of from 51 to 510 μm. Nevertheless, the manner in which such small cell openings can be achieved with a grid strut structure is not described. With respect to the strut geometry, values are given only for the strut width and strut thickness.

U.S. Pat. No. 6,129,755 discloses a stent with a grid strut structure which has several peripheral sections with 24 to 36 struts per section. The ratio of the number of struts per section to the strut length, measured in inches, is stated as >400. This means that the known stent has strut lengths in the range of from 1.5 mm to 2.3 mm. The outer diameters of the stents in question are disclosed neither in U.S. Pat. No. 6,129,755 nor in U.S. Pat. No. 6,428,569.

### BRIEF SUMMARY OF THE INVENTION

The invention is based on the object of providing a stent which has a fine grid structure and is very flexible and can be easily folded up and expanded. The stent should furthermore be comparatively easy to produce and to handle. The invention is furthermore based on the object of providing a process for the production of such a stent.

The invention is based on the concept of providing a stent with a tubular grid structure which comprises struts and cell openings formed by the struts, wherein in the expanded state of the stent the grid structure has an outer diameter D_{A} in mm and the struts each have a length L in mm. According to the invention, the ratio L/D_{A} is determined as follows:

| | |
|---|---|
| for D_{A} < 2.5 mm | L/D_{A} ≦ 0.5, in particular ≦0.4 |
| for 2.5 mm ≦ D_{A} < 4.5 mm | L/D_{A} ≦ 0.35, in particular ≦0.3 |
| for 4.5 mm ≦ D_{A} < 6 mm | L/D_{A} ≦ 0.24, in particular ≦0.21 |
| for 6 mm ≦ D_{A} < 8 mm | L/D_{A} ≦ 0.19, in particular ≦0.17 |
| for D_{A} ≧ 8 mm | L/D_{A} ≦ 0.17, in particular ≦0.16 |

The invention has the advantage that because of the short strut lengths resulting in each case from the ratio related to the particular outer diameter, the flexibility of the stent in the extended or expanded state is improved. The bending of the stent in the vessel (flexibility) is determined essentially by the regions joining the struts or generally by the regions between the struts, since the struts themselves hardly bend. Because of the short strut length, the flexibility of the stent is therefore improved.

In a preferred embodiment of the invention, the upper limit of the ratio L/D_{A} for the various diameter ranges is determined as follows:

| | |
|---|---|
| for D_{A} < 2.5 mm | L/D_{A} ≦ 0.3, in particular ≦0.2 |
| for 2.5 mm ≦ D_{A} < 4.5 mm | L/D_{A} ≦ 0.25, in particular ≦0.02 |
| for 4.5 mm ≦ D_{A} < 6 mm | L/D_{A} ≦ 0.18, in particular ≦0.15 |
| for 6 mm ≦ D_{A} < 8 mm | L/D_{A} ≦ 0.15, in particular ≦0.13 |
| for D_{A} ≧ 8 mm | L/D_{A} ≦ 0.12, in particular ≦0.10 |

In these restricted ranges, the flexibility of the stents for the various outer diameters is improved still further.

The lower limit of the ratio L/D_{A}, claimed according to a further preferred embodiment, which is determined as follows:

| | |
|---|---|
| for D_{A} < 2.5 mm | L/D_{A} ≧ 0.08, in particular ≧0.13 |
| for 2.5 mm ≦ D_{A} < 4.5 mm | L/D_{A} ≧ 0.04, in particular ≧0.08 |
| for 4.5 mm ≦ D_{A} < 6 mm | L/D_{A} ≧ 0.03, in particular ≧0.06 |
| for 6 mm ≦ D_{A} < 8 mm | L/D_{A} ≧ 0.0025, in particular ≧0.05 |
| for D_{A} ≧ 8 mm | L/D_{A} ≧ 0.0025, in particular ≧0.05 |

makes possible an economically appropriate production of the stents in various outer diameter ranges.

In a further preferred embodiment, the strut length L is determined in absolute values as follows:

| | |
|---|---|
| for D_{A} < 2.5 mm | L < 0.7 mm |
| for 2.5 mm ≦ D_{A} < 4.5 mm | 0.5 mm ≦ L ≦ 0.9 mm |
| for 4.5 mm ≦ D_{A} < 6 mm | 0.7 mm ≦ L ≦ 1.1 mm |
| for 6 mm ≦ D_{A} < 8 mm | 0.9 mm ≦ L ≦ 1.3 mm |
| for D_{A} ≧ 8 mm | 1.0 mm ≦ L < 1.5 mm |

It has been found that the abovementioned length ranges for the particular outer diameters are particularly advantageous with respect to a good flexibility of the stent.

Preferably, several peripheral sections are arranged in the longitudinal direction of the stent, wherein each peripheral section has a number N_{Steg} of struts and the ratio N_{Steg}/D_{A} is determined as follows:

| | |
|---|---|
| for D_{A} < 6 mm | N_{Steg}/D_{A} > 6, in particular ≧7 |
| for D_{A} ≧ 6 mm | N_{Steg}/D_{A} ≧ 5, in particular ≧6 |

In this context, the ratio N_{Steg}/D_{A} can be determined as follows:

| | |
|---|---|
| for D_{A} < 2.5 mm | N_{Steg}/D_{A} ≧ 9.6, in particular ≧12 |
| for 2.5 mm ≦ D_{A} < 4.5 mm | N_{Steg}/D_{A} ≧ 6.6, in particular ≧8 |
| for 4.5 mm ≦ D_{A} < 6 mm | N_{Steg}/D_{A} > 6, in particular ≧6.5 |
| for 6 mm ≦ D_{A} < 8 mm | N_{Steg}/D_{A} ≧ 6.5, in particular ≧8 |
| for D_{A} ≧ 8 mm | N_{Steg}/D_{A} ≧ 6, in particular ≧6.75 |

In absolute values, the number N_{Steg} can be determined according to the ranges as follows:

| | |
|---|---|
| for D_{A} < 2.5 mm | N_{Steg} ≧ 24 |
| for 2.5 mm ≦ D_{A} < 4.5 mm | N_{Steg} ≧ 30, in particular ≧ 36 |
| for 4.5 mm ≦ D_{A} < 6 mm | N_{Steg} > 36, in particular ≧ 40 |
| for 6 mm ≦ D_{A} < 8 mm | N_{Steg} ≧ 40, in particular ≧ 48 |
| for D_{A} ≧ 8 mm | N_{Steg} ≧ 48, in particular ≧ 54 |

The number N_{Steg} can furthermore be determined in absolute and discrete values as follows:

| | |
|---|---|
| for D_{A} < 2.5 mm | N_{Steg} = 24, 28, 30, 32 or 36 |
| for 2.5 mm ≦ D_{A} < 4.5 mm | N_{Steg} = 36 or 40 |
| for 4.5 mm ≦ D_{A} < 6 mm | N_{Steg} = 40, 42, 44 or 48 |
| for 6 mm ≦ D_{A} < 8 mm | N_{Steg} = 48, 50, 52 or 54 |
| for D_{A} ≧ 8 mm | N_{Steg} = 54 or more |

The upper limit of the ratio N_{Steg}/D_{A} can be determined as follows:

| | |
|---|---|
| for DA < 6 mm | NSteg/DA ≦ 21.6, in particular ≦ 19.2 |
| for DA > 6 mm | NSteg/DA ≦ 20, in particular ≦ 18 |

According to the invention, several peripheral sections are arranged in the longitudinal direction of the stent, each peripheral section having a number N_{Steg} of struts and the ratio N_{Steg}/L being >30, in particular >40.

The combination of short strut lengths with a large number of struts on the stent periphery leads to particularly good stent properties. On the one hand, it is therefore possible to produce stents with small cell openings, the fine grid structure having the effect of a uniform distribution of the radial force of the stent on the blood vessel or the vessel wall, since the radial force is distributed over a relatively large number of struts. As a result, vessel injuries are avoided and the neointimal hyperplasia due to locally arising forces which causes restenosis is lowered. On the other hand, this embodiment improves the flexibility still further, which makes it possible also to adapt the stent to very curved vessel sections.

According to the invention, several peripheral sections are arranged in the longitudinal direction of the stent, each peripheral section having a number N_{Steg} of struts and the ratio N_{Steg}/B being greater than or equal to 0.6, greater than or equal to 0.7, greater than or equal to 0.8, where B is the width in each case of a strut measured in μm.

The combination of a large number of struts on the periphery of the stent with small strut widths allows the construction of stents with very small cell openings and a high flexibility, which moreover can be folded up to a very small diameter for insertion into the body. The advantages described above with respect to the fine grid structure are therefore combined with an easy handling of the stent.

The cell openings each have a cell diameter D_{Z} in mm, the ratio D_{Z}/D_{A} being determined as follows:

| | |
|---|---|
| for D_{A} < 2.5 mm | D_{Z}/D_{A} ≦ 0.19, in particular ≦ 0.17 |
| for 2.5 mm ≦ D_{A} < 4.5 mm | D_{Z}/D_{A} ≦ 0.17, in particular ≦ 0.15 |
| for 4.5 mm ≦ D_{A} < 6 mm | D_{Z}/D_{A} ≦ 0.14, in particular ≦ 0.13 |
| for 6 mm ≦ D_{A} < 8 mm | D_{Z}/D_{A} ≦ 0.12, in particular ≦ 0.11 |
| for D_{A} ≧ 8 mm | D_{Z}/D_{A} ≦ 0.11, in particular ≦ 0.10 |

The lower limit of the ratio D_{Z}/D_{A} can be determined as follows:

| | |
|---|---|
| for D_{A} < 2.5 mm | D_{Z}/D_{A} ≧ 0.06, in particular ≧ 0.11 |
| for 2.5 mm ≦ D_{A} < 4.5 mm | D_{Z}/D_{A} ≧ 0.05, in particular ≧ 0.10 |
| for 4.5 mm ≦ D_{A} < 6 mm | D_{Z}/D_{A} ≧ 0.04, in particular ≧ 0.08 |
| for 6 mm ≦ D_{A} < 8 mm | D_{Z}/D_{A} ≧ 0.03, in particular ≧ 0.07 |
| for D_{A} ≧ 8 mm | D_{Z}/D_{A} ≧ 0.02, in particular ≧ 0.06 |

The advantages of correspondingly small cell diameters with respect to the distribution of the radial force of the stent have already been referred to.

Without being limited thereto, the invention is suitable in particular for stents with grid structures which comprise an open cell geometry (open cell design). The same applies to self-expanding stents.

Struts arranged adjacent to each other can be connected by an end curve or a connector in a manner known per se. In this context, the struts can be produced by laser cutting by the single slot technique in the region of the end curve or the connector and by an open cutting procedure in the remaining region of the struts. In the single slot technique, the laser beam moves slot-like between the two adjacent struts in the region of the end curve. This results in a characteristic slot-like demarcation of the two adjacent struts proximally to the end curve or to the connector. Distally from the end curve or from the connector the two adjacent struts are product by an open cutting procedure. In this distal region the struts are therefore characterized in that, in contrast to the proximal region, they are cut to form a contour. This means that the adjacent struts are each formed by contour sections which are spaced and therefore separated from one another. The stent produced in this way therefore combines stent or strut regions produced by the single slot technique and produced by an open cutting procedure.

Stents are conventionally produced either only by the single slot technique or only in an open cutting procedure.

Preferably, a gap is formed in the region of the end curve or the connector between the two struts arranged adjacent, the width of which essentially corresponds to the diameter of a laser beam used for cutting the grid structure. In this context, the end curve or the connector can have an inner radius rᵢ, wherein

| | |
|---|---|
| for D_{A} ≦ 4.5 mm | rᵢ ≦ 7 μm, in particular ≦ 5 μm, |
| | in particular ≦ 6 μm |
| for D_{A} > 4.5 mm, | rᵢ ≦ 10 μm, in particular ≦ 9 μm, |
| in particular 5 mm < D_{A} ≦ 10 mm | in particular ≦ 8 μm |

The stent produced by laser cutting partly by the single slot technique and partly by an open cutting procedure overall has the advantage that this can be folded up to a very small diameter. As a result is it possible to reach particularly small and narrow-curved vessel sections. Due to the small internal radii in particular, the adjacent struts arranged opposite can be folded up as close as possible to one another in a feed system (catheter). The combination of the single slot technique and the open cutting procedure forms an outstanding possibility for realizing the small cell openings, the short strut lengths and the relatively large number of struts on the periphery. The same applies to the small strut widths.

Preferably, extension elements can be provided, which are connected to the grid structure to decrease the size of the cell openings. The extension elements can be used as an alternative or in addition to the combined single slot/open cutting procedure for realizing the small cell openings. In this context, the extension elements can comprise tongues which are arranged between two adjacent struts. The free space between adjacent struts and therefore the cell opening are decreased in size in this way. The extension elements or tongues can be connected with end curves which connect each of two adjacent struts. In this context, the extension elements or tongues can be arranged either on the outer radius or on the inner radius of the end curves. In both cases the extension elements extend between the adjacent struts, as a result of which the intermediate space between the struts is partly covered by the extension elements or tongues. The extension elements or tongues extend essentially in the same direction as the struts, so that a uniform orientation of the struts and the extension elements is achieved.

The extension elements or tongues can each have a free end, the free end being provided at least at the same height as the end curves in the longitudinal direction of struts arranged opposite. This means that the length of the extension elements or tongues is chosen such that these extend between struts arranged opposite.

The extension elements or tongues can be angled and/or curved radially inwards and/or radially outwards. The flow dynamics in the stent are influenced by the extension elements or tongues projecting radially inwards. The extension elements or tongues projecting radially outwards improve the anchoring of the stent in the surrounding tissue.

The process according to the invention for the production of a stent, in particular of a stent as described above, is based on producing a grid structure with struts and end curves and connectors connecting these, the struts being formed by an individual slot in the proximal region of the end curves and/or connectors and by separate contours in the distal region. As a result, particularly fine structures can be produced, which can be folded up to very small diameters.

Preferably, the grid structure with struts and end curves and connectors connecting these is produced by laser cutting. In this context, the struts are formed by a single slot technique in the region of the end curves and/or connectors and by an open cutting procedure in the distal region.

The process according to this preferred embodiment combines two different cutting techniques which are used for the production of stents by means of laser cutting. In this context, stents are conventionally produced either exclusively by the single slot technique or exclusively by the open cutting procedure. In contrast, in the preferred embodiment of the process the struts are produced by the single slot technique proximally, i.e. in the region of the end curves and/or in the region of the connectors, and by the open cutting procedure distally, i.e. in the region away from the end curves or connectors. The struts are therefore slotted in the region of the end curves or connectors and cut to contour or cut out with a contour in the other strut regions away from the end curves or connectors.

The combined process makes it possible to realize very small cell openings, short and narrow struts and a large number of struts on the periphery.

It is furthermore possible, especially at thin wall thicknesses, to produce the stent by chemical or electrochemical etching.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown. In the drawings:

FIG. 1 is a plan sectional view of a segment of a stent according to an embodiment of the invention;

FIG. 2 is a segment of a grid strut structure which is produced by the single slot technique and by an open cutting procedure;

FIG. 3 is a plan sectional view of a segment of a stent having extension elements; and

FIG. 4 is a sectional view of the stent according to FIG. 3 in the folded state in a catheter.

### DETAILED DESCRIPTION OF THE INVENTION

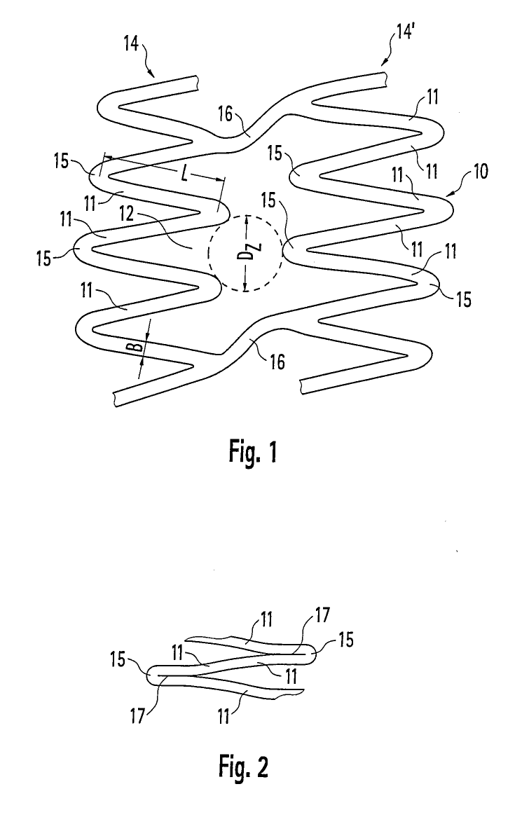

FIG. 1 shows a section (segment) of a stent according to an embodiment of the invention for the neurovascular region, in particular for treatment of aneurysms. The invention also includes stents for cerebral vessels, the preferred external diameter range being from 2.5 mm to 4.5 mm inclusive. The use of stents according to the invention not only includes the treatment of aneurysms, but is suitable generally for opening or keeping open constricted or occluded vessels.

The stent shown in section and as a diagram has a tubular grid structure 10 which comprises struts 11. The struts 11 form cell openings 12, and in particular in open cell geometry (open cell design). The invention can also be applied to stents with a closed cell design. In the expanded state, the stent has an outer diameter D_{A}. The cell diameter D_{Z} is determined by a circle with a maximum diameter which is inscribed in the strut arrangement as shown in FIG. 1. In this context, the circle inscribed touches an end curve 15 which connects two struts 11 arranged adjacent. The circle inscribed moreover touches two struts 11, or end curves 15 thereof, arranged opposite the end curve 15, so that the circle touches the strut arrangement at three points in total. Depending on the cell geometry, the circle inscribed can have another arrangement, the circle occupying the maximum possible diameter. Thus, in the case of a stent geometry where the end curves are not displaced as in FIG. 1 but are arranged directly opposite, the circle is inscribed between four adjacent struts connected with two end curves arranged opposite.

This means generally that the circle for determining the cell diameter D_{Z} is inscribed with the greatest possible diameter between struts arranged adjacent and/or end curves or connectors.

The strut length L is measured from the middle point of the end curves 15 or connectors 16 arranged at the two axial ends of a strut 11. The width B is likewise shown in FIG. 1 and relates to the stent in the expanded state.

The invention is not limited to the cell geometry shown in FIG. 1, but also includes other cell geometries.

A good flexibility of the stent is achieved if the ratio L/D_{A} is determined as follows:

| | |
|---|---|
| for D_{A} < 2.5 mm | L/D_{A} ≦ 0.5 |
| for 2.5 mm ≦ D_{A} < 4.5 mm | L/D_{A} ≦ 0.35 |
| for 4.5 mm ≦ D_{A} < 6 mm | L/D_{A} ≦ 0.24 |
| for 6 mm ≦ D_{A} < 8 mm | L/D_{A} ≦ 0.19 |
| for D_{A} ≧ 8 mm | L/D_{A} ≦ 0.17 |

By further restrictions to these ranges, i.e. by lower upper limits, the flexibility of the stents for the various outer diameters is in each case improved further, and these are disclosed as follows, the values underlined being particularly preferred:

| for D_{A} < 2.5 mm | L/D_{A} ≦ 0.45; ≦0.4; ≦0.35; ≦0.3; |
|---|---|
| | ≦0.25; ≦0.2; |
| for 2.5 mm ≦ D_{A} < 4.5 mm | L/D_{A} ≦ 0.33; ≦0.3; ≦0.27; ≦0.25; |
| | ≦0.23; ≦0.2; |
| for 4.5 mm ≦ D_{A} < 6 mm | L/D_{A} ≦ 0.21; ≦0.2; ≦0.19; ≦0.18; |
| | ≦0.17; ≦0.16; ≦0.15; |
| for 6 mm ≦ D_{A} < 8 mm | L/D_{A} ≦ 0.17; ≦0.16 ≦0.15; ≦0.14; |
| | ≦0.13; |
| for D_{A} > 8 mm | L/D_{A} ≦ 0.16; ≦0.15; ≦0.14; ≦0.13; |
| | ≦0.12; ≦0.11; ≦0.10 |

The lower limit of the ratio L/D_{A} is determined as follows:

| | |
|---|---|
| for D_{A} < 2.5 mm | L/D_{A} ≧ 0.08, in particular ≧ 0.13 |
| for 2.5 mm ≦ D_{A} < 4.5 mm | L/D_{A} ≧ 0.04, in particular ≧ 0.08 |
| for 4.5 mm ≦ D_{A} < 6 mm | L/D_{A} ≧ 0.03, in particular ≧ 0.06 |
| for 6 mm ≦ D_{A} < 8 mm | L/D_{A} ≧ 0.0025, in particular ≧ 0.05 |
| for D_{A} ≧ 8 mm | L/D_{A} ≧ 0.0025, in particular ≧ 0.05 |

Lower limits which are lower are conceivable by improved material properties, such as strength, elasticity etc., and with respect to further development of production process for production of still finer structures.

As can be seen in FIG. 1, several peripheral sections 14, 14′ are arranged in the longitudinal direction of the stent, two peripheral sections 14, 14′ being shown in the present case. In practice, more than two peripheral sections 14, 14′ are provided, each being connected to one another by connectors 16. Each peripheral section 14,14′ has a number N_{Steg} of struts 11, the ratio N_{Steg}/D_{A} being determined as follows:

| | |
|---|---|
| for D_{A} < 6 mm | N_{Steg}/D_{A} > 6, in particular ≧ 7 |
| for D_{A} ≧ 6 mm | N_{Steg}/D_{A} ≧ 5, in particular ≧ 6 |

For the various diameter ranges, the ratio N_{Steg}/D_{A} results as follows:

| | |
|---|---|
| for D_{A} < 2.5 mm | N_{Steg}/D_{A} ≧ 9.6, in particular ≧ 12 |
| for 2.5 mm ≦ D_{A} < 4.5 mm | N_{Steg}/D_{A} ≧ 6.6, in particular ≧ 8 |
| for 4.5 mm ≦ D_{A} < 6 mm | N_{Steg}/D_{A} > 6, in particular ≧ 6.5 |
| for 6 mm ≦ D_{A} < 8 mm | N_{Steg} ≧ 6.5, in particular ≧ 8 |
| for D_{A} ≧ 8 mm | N_{Steg}/D_{A} ≧ 6, in particular ≧ 6.75 |

In absolute values, the number N_{Steg} is as follows:

| | |
|---|---|
| for D_{A} < 2.5 mm | N_{Steg} ≧ 24 |
| for 2.5 mm ≦ D_{A} < 4.5 mm | N_{Steg} ≧ 30, in particular ≧ 36 |
| for 4.5 mm ≦ D_{A} < 6 mm | N_{Steg} > 36, in particular ≧ 40 |
| for 6 mm ≦ D_{A} < 8 mm | N_{Steg} ≧ 40, in particular ≧ 48 |
| for D_{A} ≧ 8 mm | N_{Steg} ≧ 48, in particular ≧ 54 |

or in discrete absolute values:

| | |
|---|---|
| for D_{A} < 2.5 mm | N_{Steg} = 24, 28, 30, 32 or 36 |
| for 2.5 mm ≦ D_{A} < 4.5 mm | N_{Steg} = 36 or 40 |
| for 4.5 mm ≦ D_{A} < 6 mm | N_{Steg} = 40, 42, 44 or 48 |
| for 6 mm ≦ D_{A} < 8 mm | N_{Steg} = 48, 50, 52 or 54 |
| for D_{A} ≧ 8 mm | N_{Steg} = 54 or more |

The upper limit of the ratio N_{Steg}/D_{A} can results as follows from the point of view of an economical production, higher upper limits being possible:

| | |
|---|---|
| for D_{A} < 6 mm | N_{Steg}/D_{A} ≦ 21.6, in particular ≦ 19.2 |
| for D_{A} > 6 mm | N_{Steg}/D_{A} ≦ 20, in particular ≦ 18 |

In the embodiment the ratios of the number of struts per peripheral section 14, 14′ to the strut length N_{Steg}/L is greater than or equal to 30 and can be greater than or equal to 40, where L is measured in mm. Further intermediate values are possible as the lower limit of the range, in particular 31, 32, 33, 34, 35, 36, 37, 38, 39 and 41, 42, 43, 44, 45, 46, 47, 48, 49, 50, 51, 52, 53, 54. In the case of strut lengths measured in μm, the abovementioned values are to be divided by the factor of 1,000. Fine-mesh grid structures which have a good flexibility are made possible by the ratio N_{Steg}/L in the ranges states. Further possible lower limits are N_{Steg}/L≧55, ≧60, ≧65, ≧70,≧75, ≧80, ≧85, ≧90. Range upper limits which are possible are N_{Steg}/L≦160, ≦155, ≦150, ≦145, ≦140, ≦135, ≦120, ≦115, ≦110, ≦105, ≦100, ≦95. In this context, the intermediate values between the abovementioned lower and upper limits are each disclosed as follows, for example: N_{Steg}/L=56, 57, 58, 59 or, respectively, N_{Steg}/L=159, 158, 157, 156. Corresponding statements apply to all the other abovementioned limit values. The values for the upper limits can in each case be combined with the values for the lower limits. In a concrete example, N_{Steg}=50 and L=0.3 mm, N_{Steg}=36 and L=0.7 or 0.4 mm.

The ratio of the number of struts per peripheral section 14, 14′ to the strut width N_{Steg}/B in the embodiment is greater than or equal to 0.6 and can be greater than or equal to 0.8, where B is the width in each case of a strut 11 measured in μm. Here also, further intermediate values are possible as the lower limit of the range, such as 0.7 or 0.9.

The abovementioned ratio allows the stent to be folded up to a small diameter, so that it can be introduced into small blood vessels. Possible lower limits for the range which are furthermore disclosed for the ratio N_{Steg}/B, where B is in μm, are N_{Steg}/B≧1, ≧1.1, ≧1.2, ≧1.3, ≧1.4, each of which involves an improvement in the compliance of the stent/vessel. Possible upper limits of the range which are disclosed are the following values, which can be combined with each of the abovementioned lower limits of the range: N_{Steg}/B≦2.5, ≦2.4, ≦2.3, ≦2.2, ≦2.1, ≦2.0, ≦1.9, ≦1.8, ≦1.7, ≦1.6, ≦1.5, ≦1.4. A concrete example of an upper limit of N_{Steg}/B=2.0 is a stent with N_{Steg}=50 and B=25 μm.

The following advantages result from the fine mesh structure of the stent according to FIG. 1 specifically for the neurovascular region, in particular for treatment of aneurysms:

Compared with conventional stents, the stent has a higher number of struts on the periphery. Moreover, the length of the individual struts is significantly shorter than in the case of conventional stents. This also results in a significantly smaller cell opening. It follows from this in turn that due to its particularly fine mesh or grid structure, the stent can isolate the aneurysm from the blood stream better than the products on the market. This has the advantage on the one hand that platinum coils, which as a rule are introduced into the aneurysm after stent implantation with the aid of a microcatheter, for better obliteration thereof, cannot unintentionally enter back into the bloodstream again. On the other hand, which is even more important, when stents according to the embodiment of the invention with the fine grid structure are employed, it is even possible to obliterate the aneurysm without the conventional filling with platinum coils merely by positioning of the stent, because the particularly fine mesh structure shields the aneurysm from the bloodstream to a sufficient extent. It is therefore possible to replace the current art of interventional aneurysm treatment, that is to say positioning of a stent in front of the aneurysm and subsequent filling of the aneurysm with platinum coils via a microcatheter, solely by positioning of a stent according to the embodiment of the invention with a fine mesh structure.

If, however, the stent with the fine mesh structure is also to be employed in combination with the platinum coils, the small cell opening also is not a disadvantage, even if the cell opening is smaller than the diameter of the microcatheter. The stent struts are in fact constructed with such filigree dimensions that the surgeon can place the microcatheter at the aneurysm by bending the stent element, i.e. essentially the struts, to the side like a metal curtain and, after depositing the platinum coils in the aneurysm and withdrawing the microcatheter, and can occlude the aneurysm again in particular on the basis of the superelastic properties of the material. This is not the case with known stents because with these the coils are positioned directly through the large cell openings.

However, the use of the stent according to the invention for cerebral vessels is not limited to only the abovementioned treatment of aneurysms, but the stent design is generally suitable for opening or keeping open constricted or occluded vessels.

The stent design according to the embodiment of the invention is particularly suitable, without being limited thereto, for self-expanding stents. In this context the short strut length is to emphasized in particular. The short strut length and, in combination with this, the small cell opening have the advantage that the aneurysm is isolated from the blood flow. Moreover, the small cell opening promotes rapid endothelialization of the narrow mesh network, as a result of which the mesh width is reduced further, since the grid structure is covered with endothelial cells. A high flexibility of the stent is furthermore ensured by the short strut length.

As the stent diameter D_{A} increases, a larger number N_{Steg} of struts 11 is positioned on the periphery. For this reason and in order to ensure that the stents can be folded up into small diameters, the struts have small strut widths B.

FIG. 2 shows a further particularly preferred embodiment of the invention, which is explained with the aid of a section of the grid structure in the region between two end curves 15. The invention is not limited to the use of end curves 15, but can also be used on connectors 16 and the associated struts 11. As can be seen in FIG. 2, two adjacently arranged struts 11 form a gap 17 in the region close to the end curve 15. In this context, the individual slot or gap 17 extends to the end curve 15 between the two struts 11. The width of the gap 17 essentially corresponds to the diameter of the laser beam used for cutting the grid structure 10. In the finished stent the gap between the struts 11 is somewhat larger than the laser beam used during curing, since material is worn away by subsequent working steps, such as electropolishing, so that the gap 17 of the finished stent is a few μm larger than the diameter of the laser beam.

The gap 17 is generated during the laser cutting by the laser beam cutting in a slot-like manner into the tubular or flat material in the region of the end curve 15. That is to say, the laser beam merely moves into the material, but does not cut out a structure. A single slot design or an individual slot which extends to the end curve 15 thereby results in the region of the end curve 15 (proximal).

The end curve 15 has an inner radius rᵢ of ≦7 μm, in particular ≦6 μm, in particular ≦5 μm for stents with an outer diameter D_{A}≦4.5 mm. For stents with an outer diameter range of 4.5 mm<D_{A}≦10 mm, the inner radius rᵢ is ≦10 μm, in particular ≦9 μm, in particular ≦8 μm.

Distally from the end curve 15, the two adjacent struts 11 are cut open or produced by an open cutting procedure, the laser beam cutting out the contour or the stent profile. The stent according to FIG. 2 therefore combines strut regions cut by the single slot technique and strut regions produced by the open cutting procedure. The single slot regions can be ⅓-½ of the strut length.

Overall, the combined regions offer the advantage that the stent can be folded up or crimped to very small diameters in the feed system, that is to say the catheter, as a result of which it is possible to reach particularly small and narrow-curved vessel sections. At the same time, the stent has good properties with respect to producibility and handling during the production process. Because of the open cutting procedure, the struts can be constructed in a large number of particular forms, angles with respect to one another etc., since due to the open cutting procedure appropriate space is available.

Both a stent which has the features which can be achieved by the combined single slot and open laser cutting, in particular in the region of the end curves 15 and connectors 16, and the abovementioned combined production process are claimed.

The stent is preferably produced by laser cutting of tubular material. In addition, the use of flat material is also possible. The stent according to FIG. 1 can also be produced by photochemical etching or chemical etching. If a flat material is used for the production, shaping of the structured flat material to the tubular design can be carried out by a heat treatment. If required, this is supplemented by a bonding technique, as a result of which the ends of the structured flat material are bonded firmly to one another. If this is to be by positive locking, laser welding is preferred for the bonding.

Shape memory alloys, for example nitinol, are used as the material.

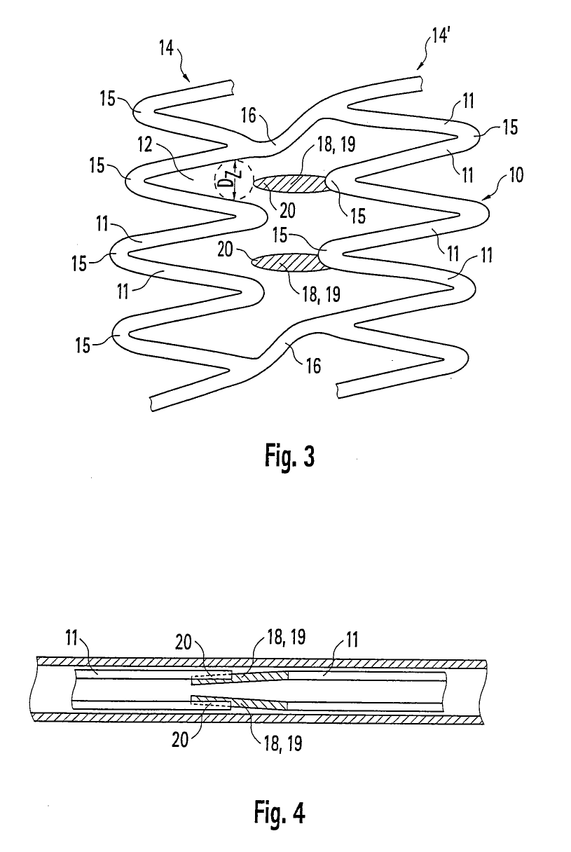

In the embodiment according to FIG. 3, extension elements 18 in the form of elongated or rod-shaped tongues 19 are provided, which are arranged at least in sections between struts 11 arranged adjacent and in this way reduce the cell diameter D_{Z}. In this context, the tongues 19 are connected to the end curves 15 and extend essentially in the same direction as the struts 11, i.e. in the longitudinal direction of the stent. In this context, the tongues each have a free end 20 which is at least at the same level as the proximal end curves 15 of the struts 11 arranged opposite in the longitudinal direction. Concretely, the free end 20 projects in between the oppositely arranged struts 11, as a result of which the distance between two peripheral sections 14, 14′ arranged in series is decreased locally.

It is also possible to arrange the tongues 19 on the inner radius of an end curve 15, so that the particular tongue 19 is arranged between the struts 11 of the same peripheral section 14, 14′. In the case of the cell geometry according to FIG. 3, the tongues 19 are arranged between the struts of a peripheral section 14, 14′ arranged before and after. In both cases the cell diameter D_{Z} is decreased.

The extension elements 18 arranged on the end curves 15 improve the flow properties of the stent in that the flow does not rip away or rips away less rapidly on the end curves 15 (tips).

When the stent is folded up, the extension elements 18 are deflected radially inwardly and are therefore moved out of the sheath surface of the stent. Alternatively, the extension elements 18 can be deflected radially outwardly and accordingly project outwardly over the sheath surface of the stent. The folded up state of the stent achieved by crimping into a feed system, for example into a catheter tube, is shown in FIG. 4. It can be seen there that the extension elements 18 are arranged partly under other stent elements or under the struts 11. By folding up the entire stent elements in more than one plane, for the same compressed diameter more surfaces than hitherto can be achieved in the expanded state.

The extension elements 18 can be arranged at a tilt or angle with respect to the sheath surface of the stent or the wall element. In this context, the extension elements 18 can each be angled downwardly, i.e. radially inwardly towards the stent central axis, or upwardly, i.e. radially outwardly away from the stent central axis. The angled extension elements can be combined with straight or likewise angled struts 11. The extension elements 18 are flexible, so that the original cell openings covered in the implanted state can easily be moved to the side by a microcatheter during so-called “coiling,” i.e., during filling of the aneurysms by coils, so that the meshes of the stent are exposed. The cell openings 12 or hollow spaces are therefore accessible. After positioning of the coils, the extension elements 18 are moved back, or are restored themselves on the bases of their elastic properties, into the original form. The aneurysm is therefore occluded again.

The embodiment according to FIG. 3, in particular the features of claim 20, are claimed both in connection with the short strut lengths L and also independently of these, since the extension elements 18 also lead per se, i.e. independently of the strut length 18, to small cell openings 12.

Concrete examples of stents according to the invention are explained in the following.

### Example 1

It can be seen from diagram 1 that the strut lengths L (strut length measured in μm) of the stents according to the invention, based on the particular outer diameter D_{A} (outer diameter measured in mm) are shorter than the particular relative strut lengths of the comparison examples. It can furthermore be seen from Diagram 1 that the ratio L/D_{A} decreases as the outer diameters D_{A} increase. Concretely, the stents according to an embodiment of the invention have the following values, the converted values furthermore being stated for strut lengths L measured in mm:

| Outer diameter | L (μm)/D_{A} (mm) | L (mm)/D_{A} (mm) |
|---|---|---|
| D_{A} = 2.5 mm | 260 | 0.26 |
| D_{A} = 3.5 mm | 205 | 0.205 |
| D_{A} = 4.5 mm | 177 | 0.177 |
| D_{A} = 6 mm | 175 | 0.175 |
| D_{A} = 8 mm | 131 | 0.131 |
| D_{A} = 8 mm | 155 | 0.155 |

It can be seen from Diagram 2 that the strut lengths L (strut length measured in μm) of the stents according to the invention are significantly shorter than the strut lengths L of the comparison stents, the strut lengths L increasing in the stents according to the invention as the stent diameter D_{A} increases. The stents according to an embodiment of the invention have the following absolute strut lengths L (in μm and mm):

| Outer diameter | Length (μm) | Length (mm) |
|---|---|---|
| D_{A} = 2.5 mm | 650 | 0.65 |
| D_{A} = 3.5 mm | 720 | 0.72 |
| D_{A} = 4.5 mm | 800 | 0.8 |
| D_{A} = 6 mm | 1,050 | 1.05 |
| D_{A} = 8 mm | 1,050 | 1.05 |
| D_{A} = 8 mm | 1,200 | 1.2 |

### Example 2

According to Diagram 3, the ratio N_{Steg}/D_{A} of the number of struts N_{Steg} to the outer diameter D_{A} (outer diameter measured in mm) in the stents according to the invention is greater than in comparison stents. It can furthermore be seen in diagram 3 that in the stents according to the invention the ratio N_{Steg}/D_{A} decreases with increasing outer diameters. The stents according to the an embodiment of the invention have the following values:

| Outer diameter | N_{Steg}/D_{A} (mm) |
|---|---|
| D_{A} = 2.5 mm | 14.4 |
| D_{A} = 3.5 mm | 10.2 |
| D_{A} = 4.5 mm | 8 |
| D_{A} = 6 mm | 8 |
| D_{A} = 8 mm | 6 |
| D_{A} = 8 mm | 6.75 |

In Diagram 4 the number of struts N_{Steg} per peripheral section is plotted against the stent diameter D_{A} (stent diameter measured in mm), where it can be seen that for all the outer diameters D_{A} the stents according to the invention have more struts on the periphery than the corresponding comparison stents. In this context, the stents according to an embodiment of the invention have the following values:

| | Number of struts per |
|---|---|
| Outer diameter | peripheral section |
| D_{A} = 2.5 mm | N_{Steg} = 36 |
| D_{A} = 3.5 mm | N_{Steg} = 36 |
| D_{A} = 4.5 mm | N_{Steg} = 36 |
| D_{A} = 6 mm | N_{Steg} = 48 |
| D_{A} = 8 mm | N_{Steg} = 48 |
| D_{A} = 8 mm | N_{Steg} = 54 |

### Example 3

In Diagram 5 the ratio N_{Steg}/L of the number of struts N_{Steg} to the strut length L (strut length measured in μm) is plotted against the outer diameter D_{A} (outer diameter measured in mm). It can be seen here that the ratio N_{Steg}/L in the stents according to the invention is significantly greater than in the comparison stents. The values for the stents according to an embodiment of the invention are as follows, the converted values for the strut length L measured in mm furthermore being stated:

| Outer diameter | N_{Steg}/L (μm) | N_{Steg}/L (mm) |
|---|---|---|
| D_{A} = 2.5 mm | 0.055 | 55 |
| D_{A} = 3.5 mm | 0.05 | 50 |
| D_{A} = 4.5 mm | 0.045 | 45 |
| D_{A} = 6 mm | 0.046 | 46 |
| D_{A} = 8 mm | 0.04 | 40 |
| D_{A} = 8 mm | 0.051 | 51 |

### Example 4

It can be seen in Diagram 6 that the ratio N_{Steg}/B of the number of struts to the strut width (strut width measured in μm) in the stents according to the invention is significantly greater than in the comparison stents. This applies in particular to the stents with the outer diameter D_{A}=2.5 mm to 4.5 mm. In this context, the stents according to an embodiment of the invention have the following values:

| Outer diameter | N_{Steg}/B (μm) | N_{Steg}/B (mm) |
|---|---|---|
| D_{A} = 2.5 mm | 1.44 | 1,440 |
| D_{A} = 3.5 mm | 1.44 | 1,440 |
| D_{A} = 4.5 mm | 1.44 | 1,440 |
| D_{A} = 6 mm | 0.8 | 800 |
| D_{A} = 8 mm | 0.8 | 800 |
| D_{A} = 8 mm | 0.9 | 900 |

In Diagram 7 the ratio D_{Z}/D_{A} between the cell opening diameter D_{Z} (cell opening diameter measured in mm) and the outer diameter D_{A} (outer diameter measured in mm) is stated, this being smaller in the stents according to the invention than in the comparison stents. In this context the values for the stents according to an embodiment of the invention are as follows:

| Outer diameter | D_{Z} (mm)/D_{A} (mm) |
|---|---|
| D_{A} = 2.5 mm | 0.14 |
| D_{A} = 3.5 mm | 0.15 |
| D_{A} = 4.5 mm | 0.14 |
| D_{A} = 6 mm | 0.128 |
| D_{A} = 8 mm | 0.11 |
| D_{A} = 8 mm | 0.126 |

In Diagram 8 the absolute values for the cell opening diameters D_{Z} (measured in mm) are plotted against the outer diameter D_{A} (outer diameter measured in mm). It can likewise be seen in diagram 8 that the cell openings in the stents according to the invention are smaller than in the comparison stents. The diameter values D_{Z} for the stents according to an embodiment of the invention are as follows:

| Outer diameter | Cell opening diameter D_{Z} (mm) |
|---|---|
| D_{A} = 2.5 mm | 0.35 |
| D_{A} = 3.5 mm | 0.526 |
| D_{A} = 4.5 mm | 0.64 |
| D_{A} = 6 mm | 0.77 |
| D_{A} = 8 mm | 0.87 |
| D_{A} = 8 mm | 0.97 |

In the context of the invention, the following features and embodiments are also disclosed:
1. Stent with a tubular grid structure (10) which comprises struts (11) and cell openings (12) formed by the struts (11), wherein in the expanded state of the stent the grid structure (10) has an outer diameter D_{A} in mm and the struts (11) each have a length L in mm,
   characterized in that
   the ratio L/D_{A} is determined as follows:

| | |
|---|---|
| for D_{A} < 2.5 mm | L/D_{A} ≦ 0.5, in particular ≦0.4 |
| for 2.5 mm ≦ D_{A} < 4.5 mm | L/D_{A} ≦ 0.35, in particular ≦0.3 |
| for 4.5 mm ≦ D_{A} < 6 mm | L/D_{A} ≦ 0.24, in particular ≦0.21 |
| for 6 mm ≦ D_{A} < 8 mm | L/D_{A} ≦ 0.19, in particular ≦0.17 |
| for D_{A} ≧ 8 mm | L/D_{A} ≦ 0.17, in particular ≦0.16 |

2. Stent according to embodiment 1,
   characterized in that
   the ratio L/DA is determined as follows:

| | |
|---|---|
| for D_{A} < 2.5 mm | L/D_{A} ≦ 0.3, in particular ≦0.2 |
| for 2.5 mm ≦ D_{A} < 4.5 mm | L/D_{A} ≦ 0.25, in particular ≦0.2 |
| for 4.5 mm ≦ D_{A} < 6 mm | L/D_{A} ≦ 0.18, in particular ≦0.15 |
| for 6 mm ≦ D_{A} < 8 mm | L/D_{A} ≦ 0.15, in particular ≦0.13 |
| for D_{A} ≧ 8 mm | L/D_{A} ≦ 0.12, in particular ≦0.10 |

3. Stent according to embodiment 1,
   characterized in that
   the lower limit of the ratio L/DA is determined as follows:

| | |
|---|---|
| for D_{A} < 2.5 mm | L/D_{A} ≧ 0.08, in particular ≧0.13 |
| for 2.5 mm ≦ D_{A} < 4.5 mm | L/D_{A} ≧ 0.04, in particular ≧0.08 |
| for 4.5 mm ≦ D_{A} < 6 mm | L/D_{A} ≧ 0.03, in particular ≧0.06 |
| for 6 mm ≦ D_{A} < 8 mm | L/D_{A} ≧ 0.0025, in particular ≧0.05 |
| for D_{A} ≧ 8 mm | L/D_{A} ≧ 0.0025, in particular ≧0.05 |

4. Stent according to at least one of embodiments 1-3
   characterized in that
   the strut length L is as follows:

| | |
|---|---|
| for D_{A} < 2.5 mm | L < 0.7 mm |
| for 2.5 mm ≦ D_{A} < 4.5 mm | 0.5 mm ≦ L ≦ 0.9 mm |
| for 4.5 mm ≦ D_{A} < 6 mm | 0.7 mm ≦ L ≦ 1.1 mm |
| for 6 mm ≦ D_{A} < 8 mm | 0.9 mm ≦ L ≦ 1.3 mm |
| for D_{A} ≧ 8 mm | 1.0 mm ≦ L < 1.5 mm |

5. Stent according to at least one of embodiments 1-4,
   characterized in that
   several peripheral sections (14, 14′) are arranged in the longitudinal direction of the stent, wherein each peripheral section (14, 14′) has a number N_{Steg} of struts (11) and the ratio N_{Steg}/D_{A} is determined as follows:

| | |
|---|---|
| for D_{A} < 6 mm | N_{Steg}/D_{A} > 6, in particular ≧7 |
| for D_{A} ≧ 6 mm | N_{Steg}/D_{A} ≧ 5, in particular ≧6 |

6. Stent according to embodiment 5,
   characterized in that
   the ratio N_{Steg}/D_{A} is determined as follows:

| | |
|---|---|
| for D_{A} < 2.5 mm | N_{Steg}/D_{A} ≧ 9.6, in particular ≧12 |
| for 2.5 mm ≦ D_{A} < 4.5 mm | N_{Steg}/D_{A} ≧ 6.6, in particular ≧8 |
| for 4.5 mm ≦ D_{A} < 6 mm | N_{Steg}/D_{A} > 6, in particular ≧6.5 |
| for 6 mm ≦ D_{A} < 8 mm | N_{Steg}/D_{A} ≧ 6.5, in particular ≧8 |
| for D_{A} ≧ 8 mm | N_{Steg}/D_{A} ≧ 6, in particular ≧6.75 |

7. Stent according to embodiment 5 or 6,
   characterized in that
   the number N_{Steg} is determined as follows:

| | |
|---|---|
| for D_{A} < 2.5 mm | N_{Steg} ≧ 24 |
| for 2.5 mm ≦ D_{A} < 4.5 mm | N_{Steg} ≧ 30, in particular ≧36 |
| for 4.5 mm ≦ D_{A} < 6 mm | N_{Steg} > 36, in particular ≧40 |
| for 6 mm ≦ D_{A} < 8 mm | N_{Steg} ≧ 40, in particular ≧48 |
| for D_{A} ≧ 8 mm | N_{Steg} ≧ 48, in particular ≧54 |

8. Stent according to at least one of embodiments 5-7
   characterized in that
   the number N_{Steg} is determined as follows:

| | |
|---|---|
| for D_{A} < 2.5 mm | N_{Steg} = 24, 28, 30, 32 or 36 |
| for 2.5 mm ≦ D_{A} < 4.5 mm | N_{Steg} = 36 or 40 |
| for 4.5 mm ≦ D_{A} < 6 mm | N_{Steg} = 40, 42, 44 or 48 |
| for 6 mm ≦ D_{A} < 8 mm | N_{Steg} = 48, 50, 52 or 54 |
| for D_{A} ≧ 8 mm | N_{Steg} = 54 or more |

9. Stent according to at least one of embodiments 5-8,
   characterized in that
   the upper limit of the ratio N_{Steg}/D_{A} is determined as follows:

| | |
|---|---|
| for D_{A} < 6 mm | N_{Steg}/D_{A} ≦ 21.6, in particular ≦ 19.2 |
| for D_{A} > 6 mm | N_{Steg}/D_{A} ≦ 20, in particular ≦ 18 |

10. Stent according to at least one of embodiments 1-9,
   characterized in that
   several peripheral sections (14, 14′) are arranged in the longitudinal direction of the stent, each peripheral section (14, 14′) having a number N_{Steg} of struts (11) and the ratio N_{Steg}/L being >30, in particular >40, where the strut length L is measured in mm.
11. Stent according to at least one of embodiments 1-10,
   characterized in that
   several peripheral sections (14, 14′) are arranged in the longitudinal direction of the stents, each peripheral section (14, 14′) having a number NSteg of struts (11) and the ratio NSteg/B being >0.6, in particular >0.7, where the width B of each strut (11) is measured in μm.
12. Stent according to at least one of embodiments 1-11,
   characterized in that
   the cell openings (12) each have a cell diameter D_{Z} in mm, the ratio D_{Z}/D_{A} being determined as follows:

| | |
|---|---|
| for D_{A} < 2.5 mm | D_{Z}/D_{A} ≦ 0.19, in particular ≦ 0.17 |
| for 2.5 mm ≦ D_{A} < 4.5 mm | D_{Z}/D_{A} ≦ 0.17, in particular ≦ 0.15 |
| for 4.5 mm ≦ D_{A} < 6 mm | D_{Z}/D_{A} ≦ 0.14, in particular ≦ 0.13 |
| for 6 mm ≦ D_{A} < 8 mm | D_{Z}/D_{A} ≦ 0.12, in particular ≦ 0.11 |
| for D_{A} ≧ 8 mm | D_{Z}/D_{A} ≦ 0.11, in particular ≦ 0.10 |

13. Stent according to embodiment 12,
   characterized in that
   the lower limit of the ratio D_{Z}/D_{A} is determined as follows:
   for D_{A}<2.5 mmD_{Z}/D_{A}≧0.06, in particular ≧0.11
   for 2.5 mm≦D_{A}<4.5 mmD_{Z}/D_{A}≧0.05, in particular ≧0.10
   for 4.5 mm≦D_{A}<6 mmD_{Z}/D_{A}≧0.04, in particular ≧0.08
   for 6 mm≦D_{A}<8 mmD_{Z}/D_{A}≧0.03, in particular ≧0.07
   for D_{A}≧8 mmD_{Z}/D_{A}≧0.02, in particular ≧0.06
14. Stent according to at least one of embodiments 1-13,
   characterized in that
   the grid structure (10) comprises an open cell geometry (open cell design).
15. Stent according to at least one of embodiments 1-14,
   characterized in that
   the stent is self-expanding.
16. Stent according to at least one of embodiments 1-15,
   characterized in that
   in each case two struts (11) arranged adjacent are connected by an end curve (15) or a connector (16).
17. Stent according to embodiment 16,
   characterized in that
   the struts (11) are produced by laser cutting by the single slot technique in the region of the end curve (15) or the connector (16) and by an open cutting procedure in the remaining region of the struts (11).
18. Stent according to embodiment 15 or 17,
   characterized in that
   a gap is formed in the region of the end curve (15) or the connector (16) between the two struts (11) arranged adjacent, the width of which essentially corresponds to the diameter of a laser beam used for cutting the grid structure (10).
19. Stent according to at least one of embodiments 16-18,
   characterized in that
   the end curve (15) or the connector (16) has an inner radius rᵢ, wherein

| | |
|---|---|
| for D_{A} ≦ 4.5 mm | rᵢ ≦ 7 μm, in particular ≦ 6 μm, in |
| | particular ≦ 5 μm |
| for D_{A} > 4.5 mm, in particular | rᵢ ≦ 10 μm, in particular ≦ 9 μm, |
| 4.5 mm < D_{A} ≦ 10 mm | in particular ≦ 8 μm |

20. Stent according to at least one of embodiments 1-19,
   characterized in that
   extension elements (18) are provided, which are connected to the grid structure (10) to reduce the size of the cell openings (12).
21. Stent according to embodiment 20,
   characterized in that
   the extension elements (18) comprise tongues (19) which are arranged between two adjacent struts (11).
22. Stent according to embodiment 20 or 21,
   characterized in that
   the extension elements (18) or tongues (19) are connected with end curves (15) which connect each of two struts (11) arranged adjacent.
23. Stent according to at least one of embodiments 20-22
   characterized in that
   the extension elements (18) or tongues (19) essentially extend in the same direction as the struts (11).
24. Stent according to at least one of embodiments 20-23,
   characterized in that
   the extension elements (18) or tongues (19) each have a free end (20), the free end (20) being provided at least at the same level as the end curves (15) of struts (11) arranged opposite in the longitudinal direction of the stent.
25. Stent according to at least one of embodiments 20-24,
   characterized in that
   the extension elements (18) or tongues (19) can be angled and/or curved radially inwards and/or radially outwards.
26. Process for the production of a stent, in particular of a stent according to embodiments 1-25, wherein a grid structure (10) with struts (11) and end curves (15) and connectors (16) connecting these are produced, wherein the struts (11) are formed together by an individual slot in the region of the end curves (15) and/or connectors (16) and by separate contours in the distal region.
27. Process according to embodiment 26,
   characterized in that
   struts (11) are formed by laser cutting.
28. Process according to embodiment 26,
   characterized in that
   struts (11) are formed by chemical or electrochemical etching.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1.-25. (canceled)

26. A stent having a tubular grid structure (10) which comprises struts (11) and cell openings (12) formed by the struts (11), wherein in an expanded state of the stent the grid structure (10) has an outer diameter D_{A} in mm and the struts (11) each have a length L in mm and a width B in μm, wherein a ratio L/D_{A} is determined as follows:

| | |
|---|---|
| for D_{A} < 2.5 mm | L/D_{A} ≦ 0.5, optionally ≦ 0.4 |
| for 2.5 mm ≦ D_{A} < 4.5 mm | L/D_{A} ≦ 0.35, optionally ≦ 0.3 |
| for 4.5 mm ≦ D_{A} < 6 mm | L/D_{A} ≦ 0.24, optionally ≦ 0.21 |
| for 6 mm ≦ D_{A} < 8 mm | L/D_{A} ≦ 0.19, optionally ≦ 0.17 |
| for D_{A} ≧ 8 mm | L/D_{A} ≦ 0.17, optionally ≦ 0.16 |

wherein several peripheral sections (14, 14′) are arranged in a longitudinal direction of the stent, each peripheral section (14, 14′) having a number N_{Steg} of struts (11) and a ratio N_{Steg}/L being >30, optionally >40, and a ratio N_{Steg}/B is >0.6, optionally >0.7, optionally >0.8, optionally >0.9, optionally >1, optionally >1.1, optionally >1.2, optionally >1.3, optionally >1.4, and
wherein the cell openings (12) each have a cell diameter D_{Z} in mm, a ratio D_{Z}/D_{A} being determined as follows:

| | |
|---|---|
| for D_{A} < 2.5 mm | D_{Z}/D_{A} ≦ 0.19, optionally ≦ 0.17 |
| for 2.5 mm ≦ D_{A} < 4.5 mm | D_{Z}/D_{A} ≦ 0.17, optionally ≦ 0.15 |
| for 4.5 mm ≦ D_{A} < 6 mm | D_{Z}/D_{A} ≦ 0.14, optionally ≦ 0.13 |
| for 6 mm ≦ D_{A} < 8 mm | D_{Z}/D_{A} ≦ 0.12, optionally ≦ 0.11 |
| for D_{A} ≧ 8 mm | D_{Z}/D_{A} ≦ 0.11, optionally ≦ 0.10. |

27. The stent according to claim 26, wherein the ratio L/D_{A} is determined as follows:

| | |
|---|---|
| for D_{A} < 2.5 mm | L/D_{A} ≦ 0.3, optionally ≦ 0.2 |
| for 2.5 mm ≦ D_{A} < 4.5 mm | L/D_{A} ≦ 0.25, optionally ≦ 0.2 |
| for 4.5 mm ≦ D_{A} < 6 mm | L/D_{A} ≦ 0.18, optionally ≦ 0.15 |
| for 6 mm ≦ D_{A} < 8 mm | L/D_{A} ≦ 0.15, optionally ≦ 0.13 |
| for D_{A} ≧ 8 mm | L/D_{A} ≦ 0.12, optionally ≦ 0.10. |

28. The stent according to claim 26, wherein a lower limit of the ratio L/D_{A} is determined as follows:

| | |
|---|---|
| for D_{A} < 2.5 mm | L/D_{A} ≧ 0.08, optionally ≧ 0.13 |
| for 2.5 mm ≦ D_{A} < 4.5 mm | L/D_{A} ≧ 0.04, optionally ≧ 0.08 |
| for 4.5 mm ≦ D_{A} < 6 mm | L/D_{A} ≧ 0.03, optionally ≧ 0.06 |
| for 6 mm ≦ D_{A} < 8 mm | L/D_{A} ≧ 0.0025, optionally ≧ 0.05 |
| for D_{A} ≧ 8 mm | L/D_{A} ≧ 0.0025, optionally ≧ 0.05. |

29. The stent according to claim 26, wherein the strut length L is as follows:

| | |
|---|---|
| for D_{A} < 2.5 mm | L < 0.7 mm |
| for 2.5 mm ≦ D_{A} < 4.5 mm | 0.5 mm ≦ L ≦ 0.9 mm |
| for 4.5 mm ≦ D_{A} < 6 mm | 0.7 mm ≦ L ≦ 1.1 mm |
| for 6 mm ≦ D_{A} < 8 mm | 0.9 mm ≦ L ≦ 1.3 mm |
| for D_{A} ≧ 8 mm | 1.0 mm ≦ L < 1.5 mm. |

30. The stent according to claim 26, wherein a ratio N_{Steg}/D_{A} is determined as follows:

| | |
|---|---|
| for D_{A} < 6 mm | N_{Steg}/D_{A} > 6, optionally ≧ 7 |
| for D_{A} ≧ 6 mm | N_{Steg}/D_{A} ≧ 5, optionally ≧ 6. |

31. The stent according to claim 30, wherein the ratio N_{Steg}/D_{A} is determined as follows:

| | |
|---|---|
| for D_{A} < 2.5 mm | N_{Steg}/D_{A} ≧ 9.6, optionally ≧ 12 |
| for 2.5 mm ≦ D_{A} < 4.5 mm | N_{Steg}/D_{A} ≧ 6.6, optionally ≧ 8 |
| for 4.5 mm ≦ D_{A} < 6 mm | N_{Steg}/D_{A} > 6, optionally ≧ 6.5 |
| for 6 mm ≦ D_{A} < 8 mm | N_{Steg}/D_{A} ≧ 6.5, optionally ≧ 8 |
| for D_{A} ≧ 8 mm | N_{Steg}/D_{A} ≧ 6, optionally ≧ 6.75. |

32. The stent according to claim 30, wherein the number N_{Steg} is determined as follows:

| | |
|---|---|
| for D_{A} < 2.5 mm | N_{Steg} ≧ 24 |
| for 2.5 mm ≦ D_{A} < 4.5 mm | N_{Steg} ≧ 30, optionally ≧ 36 |
| for 4.5 mm ≦ D_{A} < 6 mm | N_{Steg} > 36, optionally ≧ 40 |
| for 6 mm ≦ D_{A} < 8 mm | N_{Steg} ≧ 40, optionally ≧ 48 |
| for D_{A} ≧ 8 mm | N_{Steg} ≧ 48, optionally ≧ 54. |

33. The stent according to claim 30, wherein the number N_{Steg} is determined as follows:

| | |
|---|---|
| for D_{A} < 2.5 mm | N_{Steg} = 24, 28, 30, 32 or 36 |
| for 2.5 mm ≦ D_{A} < 4.5 mm | N_{Steg} = 36 or 40 |
| for 4.5 mm ≦ D_{A} < 6 mm | N_{Steg} = 40, 42, 44 or 48 |
| for 6 mm ≦ D_{A} < 8 mm | N_{Steg} = 48, 50, 52 or 54 |
| for D_{A} ≧ 8 mm | N_{Steg} = 54 or more. |

34. The stent according to claim 30, wherein an upper limit of the ratio N_{Steg}/D_{A} is determined as follows:

| | |
|---|---|
| for D_{A} < 6 mm | N_{Steg}/D_{A} ≦ 21.6, optionally ≦ 19.2 |
| for D_{A} > 6 mm | N_{Steg}/D_{A} ≦ 20, optionally ≦ 18. |

35. The stent according to claim 26, wherein a lower limit of the ratio D_{Z}/D_{A} is determined as follows:

| | |
|---|---|
| for D_{A} < 2.5 mm | D_{Z}/D_{A} ≧ 0.06, optionally ≧ 0.11 |
| for 2.5 mm ≦ D_{A} < 4.5 mm | D_{Z}/D_{A} ≧ 0.05, optionally ≧ 0.10 |
| for 4.5 mm ≦ D_{A} < 6 mm | D_{Z}/D_{A} ≧ 0.04, optionally ≧ 0.08 |
| for 6 mm ≦ D_{A} < 8 mm | D_{Z}/D_{A} ≧ 0.03, optionally ≧ 0.07 |
| for D_{A} ≧ 8 mm | D_{Z}/D_{A} ≧ 0.02, optionally ≧ 0.06. |

36. The stent according to claim 26, wherein the grid structure (10) comprises an open cell geometry (open cell design).

37. The stent according to claim 26, wherein the stent is self-expanding.

38. The stent according to claim 26, wherein in each case two adjacently arranged struts (11) are connected by an end curve (15) or a connector (16).

39. The stent according to claim 38, wherein the struts (11) are produced by laser cutting by a single slot technique in a region of the end curve (15) or the connector (16) and by an open cutting procedure in a remaining region of the struts (11).

40. The stent according to claim 39, wherein a gap is formed in the region of the end curve (15) or the connector (16) between the two adjacently arranged struts (11), a width of the gap essentially corresponding to a diameter of a laser beam used for cutting the grid structure (10).

41. The stent according to claim 38, wherein the end curve (15) or the connector (16) has an inner radius rᵢ, wherein

| | |
|---|---|
| for D_{A} ≦ 4.5 m | rᵢ ≦ 7 μm, optionally ≦ 6 μm, |
| | optionally ≦ 5 μm |
| for D_{A} > 4.5 mm, | rᵢ ≦ 10 μm, optionally ≦ 9 μm, |
| optionally 4.5 mm < D_{A} ≦ 10 mm | optionally ≦ 8 μm |

42. The stent according to claim 26, wherein extension elements (18) are provided connected to the grid structure (10) to reduce a size of the cell openings (12).

43. The stent according to claim 42, wherein the extension elements (18) comprise tongues (19) arranged between two adjacent struts (11).

44. The stent according to claim 43, wherein the tongues (19) are connected with end curves (15) which connect each of two adjacently arranged struts (11).

45. The stent according to claim 42, wherein the extension elements (18) essentially extend in a same direction as the struts (11).

46. The stent according to claim 42, wherein the extension elements (18) each have a free end (20), the free end (20) being provided at least at a same level as end curves (15) of struts (11) arranged oppositely in a longitudinal direction of the stent.

47. The stent according to claim 42, wherein the extension elements (18) can be angled and/or curved radially inwardly and/or radially outwardly.

48. A process for production of a stent, comprising forming a grid structure (10) having struts (11) with end curves (15) and connectors (16) connecting the struts, wherein the struts (11) are formed together by an individual slot in a region of the end curves (15) and/or connectors (16) and by separate contours in a distal region.

49. The process according to claim 48, wherein the struts (11) are formed by laser cutting.

50. The process according to claim 48, wherein the struts (11) are formed by chemical or electrochemical etching.
